(19) United States
(12) Patent Application Publication
(10) Pub. No.: US 2026/0007712 A1
(43) Pub. Date: Jan. 8, 2026

(54) COMPOSITION FOR PREVENTING, AMELIORATING OR TREATING PERIODONTAL DISEASES

(71) Applicant: NUTRACORE CO., LTD., Suwon-si (KR)

(72) Inventors: Beom Rak CHOI, Suwon-si, Gyeonggi-do (KR); Sae-Kwang KU, Suseong-gu, Daegu (KR); Hye-Rim PARK, Hwaseong-si, Gyeonggi-do (KR)

(21) Appl. No.: 18/843,522

(22) PCT Filed: Mar. 3, 2023
(86) PCT No.: PCT/KR2023/002988

§ 371 Date: Mar. 11, 2025

(30) Foreign Application Priority Data
Mar. 4, 2022 (KR) 10-2022-0028210

(51) Int. Cl. A61K 36/758; A61K 8/9789; A61K 36/31; A61P 1/02; A61Q 11/00

(52) U.S. Cl. CPC A61K 36/758; A61K 8/9789; A61K 36/31; A61P 1/02; A61Q 11/00

(57) ABSTRACT

In one aspect, a composition for preventing, ameliorating, or treating periodontal diseases includes a combination of Zanthoxylum schinifolium extract and Wasabia japonica leaf extract, which are natural extracts, as an active ingredient. The composition an anti-inflammatory effect against periodontal diseases, and exhibits the effect of preventing, ameliorating, or treating periodontal diseases by suppressing periodontal bone loss and protecting collagen in gum tissue.

### TECHNICAL FIELD

This application claims priority based on Korean Application No. 10-2022-0028210 filed on Mar. 4, 2022, and all contents disclosed in the specification and drawings of the application are incorporated by reference herein.

The present invention relates to a composition for preventing, improving or treating periodontal disease, comprising natural extract(s) as an active ingredient.

### BACKGROUND ART

Periodontal disease is a chronic inflammatory disease characterized by destruction of the connective tissue and cementum that support the teeth, bone resorption, leukocyte infiltration, and periodontal pocket formation. Periodontal disease, a cause of tooth loss in adults, is characterized by localized bone resorption. The presence of bacterial plaque has been considered a pathogenesis of associated periodontal diseases, which may induce a local inflammatory response. This inflammatory response leads to edema, leukocyte infiltration, and release of inflammatory mediators, leading to periodontal pocket formation, connective tissue detachment, and alveolar bone resorption, ultimately resulting in tooth loss. Recently, nitric oxide activity and oxidative stress have been found to be involved in the pathogenesis of periodontitis, and many antioxidants have shown beneficial effects on periodontitis and associated alveolar bone loss.

Currently, periodontitis is treated by removing plaque and tartar through scaling, or the gums may be excised to reduce the depth of periodontal pockets created between teeth and gums. If periodontitis is severe, antibiotics may be prescribed or antibiotic ointment may be placed in the periodontal pockets between the teeth and gums. However, as problems arise due to overuse of antibiotics and side effects occur due to prescriptions of chemical drugs, attempts are being made to develop various alternative treatments derived from natural products that are expected to have relatively low side effects. In particular, ingredients derived from natural products contain a variety of phenolic compounds, vitamins, carotenoids, and flavonoids, and are known to exhibit various pharmacological effects, including immune regulation and anti-inflammatory effects through antioxidant mechanisms. In this context, efforts have recently been made to find a treatment for periodontal disease from natural products that has low side effects and relatively strong anti-inflammatory effects. Currently, various natural products are known to regulate the body's immune system and exhibit antioxidant and anti-inflammatory effects, and in particular, various natural extracts have been reported to have treatment or inhibitory effects on periodontal disease and inflammation.

Numerous documents are referenced and cited throughout this specification. The disclosures of the cited documents are incorporated herein by reference in their entirety to more clearly explain the content of the present invention and the level of technical field to which the present invention pertains.

### DISCLOSURE

### Technical Problem

The present inventors have made efforts to develop natural products that are effective in preventing, improving, and treating periodontal diseases such as gingivitis and periodontitis while having no side effects. As a result, this invention has been completed by discovering that Sancho (Zanthoxyli Pericarpium) extract and wasabi leaf extract have particularly excellent effects in preventing, improving, or treating periodontal disease among the various natural extracts known for their antioxidant and anti-inflammatory effects, and their combination is particularly effective in preventing, improving, or treating periodontal disease since there is a significant synergistic effect compared to the effect of each extract.

Accordingly, the purpose of the present invention is to provide the following embodiments.
Embodiment 1. A composition for preventing, improving or treating periodontal disease comprising a combination of Sancho extract (ZP) and wasabi leaf extract (WF) as an active ingredient; a use of a combination of Sancho extract (ZP) and wasabi leaf extract (WF) for the manufacture of products (compositions, foods, health functional foods, pharmaceuticals and/or quasi-drugs) for preventing, improving or treating periodontal disease; or a method for preventing, improving or treating periodontal disease, comprising applying a composition comprising a combination of Sancho extract (ZP) and wasabi leaf extract (WF) as an active ingredient to a subject in need thereof.
Embodiment 2. The composition; use; or method according to Embodiment 1, wherein the weight ratio of the Sancho extract to the wasabi leaf extract is 1:4 to 8:1 (ZP:WF).
Embodiment 3. The composition; use; or method according to any one of the preceding embodiments, wherein the weight ratio of the Sancho extract to the wasabi leaf extract is 1:2 to 4:1 (ZP:WF).
Embodiment 4. The composition; use; or method according to any one of the preceding embodiments, wherein the weight ratio of the Sancho extract to the wasabi leaf extract is 1:2 to 2:1 (ZP:WF).
Embodiment 5. The composition; use; or method according to any one of the preceding embodiments, wherein the wasabi leaf extract contains 1.0 to 27.7 mg/g of sinigrin or 0.1 to 3.0 mg/g of isovitexin.
Embodiment 6. The composition; use; or method according to any one of the preceding embodiments, wherein the extract is extracted using any one selected from the group consisting of water, lower alcohols having 1 to 4 carbon atoms, and mixtures thereof as an extraction solvent.
Embodiment 7. The composition; use; or method according to any one of the preceding embodiments, wherein the periodontal disease is gingivitis or periodontitis.
Embodiment 8. The composition; use; or method according to any one of the preceding embodiments, wherein the Sancho extract contains 0.5 to 10.0 mg/g of syringin.
Embodiment 9. The composition; use; or method according to any one of the preceding embodiments, the composition is a food composition for preventing or improving periodontal disease.
Embodiment 10. The composition; use; or method according to any one of the preceding embodiments, wherein the composition is a pharmaceutical composition.
Embodiment 11. A health functional food comprising the composition according to any one of the preceding embodiments.
Embodiment 12. The composition; use; or method according to any one of the preceding embodiments, wherein the composition is a toothpaste composition.

Further objects and advantages of the present invention will become clearer from the following detailed description, claims, and drawings.

### Technical Solution

One aspect of the present invention is to provide a composition for preventing, improving or treating periodontal disease comprising a combination of Sancho extract and wasabi leaf extract as an active ingredient.

Sancho (Zanthoxyli Pericarpium) is an herbal medicine that has been used for a long time as a medicinal herb made from the dried bark of the ripe fruit of the Zanthoxylum schinifolium Siebold & Zucc. Sancho is described in Donguibogam as having anthelmintic and sterilizing effects, and is also known to be effective in treating indigestion. In one embodiment, the Sancho extract of the present invention contains 0.5 to 10.0 mg/g of syringin as an active ingredient, and it has been confirmed by the present inventors to be effective in preventing, improving, or treating periodontal diseases such as gingivitis and periodontitis by inhibiting periodontal bone loss and protecting collagen in gum tissue.

Wasabi (Wasabia/Eutrema japonica Matsum.) is a native Japanese plant that has been used medicinally for a long time. It contains a large amount of isothiocyanate compounds such as allyisothiocyanate, and has a unique irritating taste, so it has been used as a spice or pickle. Although the antioxidant and anticancer effects of wasabi root are well known, the physiological activities of parts other than wasabi root, especially the leaves, are not well known. In one embodiment, the wasabi leaf extract of the present invention contains 1.0 to 27.7 mg/g of sinigrin or 0.1 to 3.0 mg/g of isovitexin as an active ingredient, and it has been confirmed by the present inventors to be effective in preventing, improving, or treating periodontal diseases such as gingivitis and periodontitis by inhibiting periodontal bone loss and protecting collagen in gum tissue.

The composition of the present invention may further comprise Rehmanniae radix extract as an active ingredient in addition to the Sancho extract and wasabi leaf extract.

Jihwang (Rehmanniae radix) is the root of Rehmannia glutinosa (Gaertn.) Libosch. ex Steud. When the root is used as is, it is called raw Jihwang; when the root is dried and used, it is called dried Jihwang; and when steamed with alcohol, it is called aged Jihwang. It is known to contain catalpol and mannit as medicinal ingredients, and mannitol, stachyose, glucose, etc. in the water-soluble portion. Raw Jihwang extract promotes blood coagulation and increases the contractility of the heart muscle in a weakened heart. Additionally, it has diuretic and antipyretic effects. Aged Jihwang is recognized as having a nourishing and tonic effect, lowering blood sugar levels, and having a tonic and diuretic effect. Aged Jihwang is highly effective as a treatment for anemia, and is also effective in improving decline in reproductive function, chronic nephritis, high blood pressure, diabetes, and nervous breakdown. In addition, it is widely used for relieving kidney function weakness and asthma. Raw Jihwang is cold and bitter and sweet in taste, dried Jihwang is cold and sweet, and aged Jihwang is slightly mild and sweet.

The composition of the present invention comprises extracts of the above natural ingredients as an active ingredient. The term ‘extract’ used herein refers to the extraction result obtained by extract-juicing the raw material or treating the raw material with an extraction solvent, or is meant to include processed products obtained by formulating (e.g., powdering) the extraction result.

When the extract used in the composition of the present invention is obtained by treating the raw material with an extraction solvent, various extraction solvents, for example, a polar solvent or a non-polar solvent can be used. As the polar solvent, (i) water, (ii) alcohol (preferably methanol, ethanol, propanol, butanol, n-propanol, iso-propanol, n-butanol, 1-pentanol, 2-butoxyethanol, or ethylene glycol), (iii) acetic acid, (iv) DMF (dimethyl-formamide), and (v) DMSO (dimethyl sulfoxide), etc. can be used. As the non-polar solvents, acetone, acetonitrile, ethyl acetate, methyl acetate, fluoroalkane, pentane, hexane, 2,2,4-trimethylpentane, decane, cyclohexane, cyclopentane, diisobutylene, 1-pentene, 1-chlorobutane, 1-chloropentane, o-xylene, diisopropyl ether, 2-chloropropane, toluene, 1-chloropropane, chlorobenzene, benzene, diethyl ether, diethyl sulfide, chloroform, dichloromethane, 1,2-dichloroethane, aniline, diethylamine, ether, carbon tetrachloride and THF, etc. can also be used.

Preferably, the extract used in the present invention may be extracted by squeezing the raw material or using any one selected from the group consisting of water, lower alcohols with 1 to 4 carbon atoms, and mixtures thereof as an extraction solvent, but is not limited thereto.

In addition, the term ‘extract’ used in this specification has the meaning commonly used in the art as a crude extract, as described above, but in a broad sense also includes fractions obtained by additional fractionation of the extract. That is, it includes not only extracts obtained by squeezing the raw material or using the above-mentioned extraction solvent, but also those obtained by additionally applying a purification process. For example, fractions obtained by passing the extract through an ultrafiltration membrane with a certain molecular weight cut-off value, separation by various chromatographs (designed for separation according to size, charge, hydrophobicity, or affinity), etc., and fractions obtained through purification methods are also included in the extract of the present invention.

In addition, the extract of the present invention may be one in which the solvent is removed by performing additional processes, such as filtration, concentration, or drying, or all of filtration, concentration, and drying. For filtration, for example, filter paper or a vacuum filter can be used. For concentration, a vacuum concentrator can be used. For drying, a powdered extract can be obtained by performing spray drying or freeze-drying.

In the composition of the present invention, the active ingredient may be a combination of Sancho extract and wasabi leaf extract, and when combined, it is characterized by a synergistic effect compared to the group administered alone. More specifically, the weight ratio of the Sancho extract to the wasabi leaf extract is 1:4 to 8:1 (ZP:WF), for example, ZP:WF=1:4, 1:2, 1:1, 2:1, 4:1 and 8:1, preferably in a weight ratio of 1:2 to 4:1 (ZP:WF), more preferably in a weight ratio of 1:2 to 2:1 (ZP:WF), but is not limited thereto.

The active ingredient may be included in an amount of 0.1 to 5% by volume based on the total volume of the composition of the present invention. The active ingredient has an anti-inflammatory effect on periodontal disease, and indicates the effect of preventing, improving or treating periodontal disease by suppressing periodontal bone loss and protecting collagen in gum tissue.

In one embodiment, the composition of the present invention may be a food composition. The food composition of the present invention includes all processed forms of natural materials such as foods, functional foods, nutritional supplements, health feeds, and food additives. Food compositions of this type can be prepared in various forms according to conventional methods known in the art.

For example, as a health food, the extract of the present invention itself can be prepared and consumed in the form of tea, juice, and drinks, or can be consumed by granulating, encapsulating, or powdering. In addition, in addition to the extract of the present invention, dried root of Paeonia japonica, fruits of Cornus officinalis, ezo-ukogi, lingzhi mushroom, dried tangerine peel, cordyceps, Angelica sinensis, Gardenia fruit, astragalus, malted barley, trifoliate orange, vitamin C, fructo-oligosaccharide, stevioside, purified water, maltodextrin, etc. may be further included alone or in combination within the range of not inhibiting the purpose of the present invention, but other medicinal ingredients and/or additives that may be additionally included in the food composition of the present invention are not limited to the above examples.

For example, the food composition according to the present invention may include thiamine (vitamin B1), riboflavin, ascorbic acid, niacin, and vitamin B6 as water-soluble vitamins; myristic acid, palmitic acid, stearic acid, oleic acid, linoleic acid, etc. as fatty acids; glycolic acid and acetic acid as weak acid components; and threonine, valine, methionine, isoleucine, leucine, phenylalanine, tryptophan, and lysine as eight essential amino acids, and aspartic acid, serine, glutamic acid, proline, glycine, alanine, cysteine, tyrosine, histidine, arginine, etc. as other amino acids.

In another embodiment, the composition of the present invention may be a pharmaceutical composition. The pharmaceutical composition comprises the above-mentioned active ingredients and can be formulated into a pharmaceutical unit dosage form by adding pharmaceutically acceptable carriers, excipients, or diluents.

The term “pharmaceutically acceptable” refers to a non-toxic ingredient or composition that is physiologically acceptable and does not inhibit the action of the active ingredient and does not usually cause gastrointestinal disorders, allergic reactions such as dizziness, or similar reactions when administered to humans.

Examples of such carriers, excipients or diluents include lactose, dextrose, sucrose, sorbitol, mannitol, xylitol, erythritol, maltitol, starch, gum acacia, alginate, gelatin, calcium phosphate, calcium silicate, cellulose, methyl cellulose, polyvinylpyrrolidone, water, methylhydroxybenzoate, propylhydroxybenzoate, talc, magnesium stearate, and mineral oil. Additionally, the pharmaceutical composition may further include fillers, anti-coagulants, lubricants, wetting agents, flavorings, emulsifiers, or preservatives.

The term “pharmaceutically effective amount” refers to an amount that shows a greater response than the negative control, and preferably refers to an amount sufficient to show the effect of preventing and/or treating periodontal disease.

Additionally, the pharmaceutical compositions of the present invention can be formulated using methods known in the art to provide rapid, sustained or delayed release of the active ingredient after administration to a mammal. The dosage form may be, for example, selected from the group consisting of gel, paste, ointment, powder, emulsion, spray and aerosol, but is not limited thereto.

In addition, the pharmaceutical composition of the present invention can be administered in combination with a known compound having a preventive and/or therapeutic effect for periodontal disease.

In another embodiment, the composition of the present invention may be a toothpaste composition.

The toothpaste composition of the present invention may further include other additives commonly used in toothpaste compositions in the art, depending on the formulation and purpose of use, to the extent that they do not impair the purpose of the present invention. The other additives may further include, for example, abrasives, flavoring agents, sweeteners, medicinal agents, pigments, pH adjusters, preservatives, and whitening agents.

### Advantageous Effects

The composition of the present invention exerts an anti-inflammatory effect on periodontal disease and prevents, improves or treats periodontal disease by inhibiting periodontal bone loss and protecting collagen in gum tissue.

### BRIEF DESCRIPTION OF DRAWINGS

FIG. 1 shows the Sancho extract prepared in Example.

FIG. 2 shows the wasabi leaf extract prepared in the example.

FIG. 3 shows the results of measuring changes in periodontal bone loss scores (Alveolar bone loss scores).

FIG. 4 shows the results of measuring changes in periodontal bone volume (Alveolar bone volume).

FIG. 5 shows the results of measuring the number of inflammatory cells in gum tissue.

FIG. 6 shows the results of measuring the collagen fiber content in the gums.

FIG. 7 shows the results of measuring the activity of inhibiting the expression of inflammatory cytokines in the gums.

FIG. 8 shows the results of measuring the activity of inhibiting MDA expression in the gums.

FIG. 9 shows the results of measuring the change in periodontal bone loss rate (Alveolar bone loss) when administering Sancho extract (ZP) and wasabi leaf extract (WF) composites compared to when each was administered alone.

FIG. 10 shows the results of measuring the change in periodontal bone volume (Alveolar bone volume) when administering Sancho extract (ZP) and wasabi leaf extract (WF) composites compared to when each was administered alone.

### MODE FOR INVENTION

Hereinafter, the present invention will be described in more detail through examples. These examples are only for illustrating the present invention in more detail, and it will be apparent to those skilled in the art that the scope of the present invention is not limited by these examples.

### Example

### I. Preparation of Test Substances and Confirmation of Indicator Components

Sancho (ZP) extract: Zanthoxyli Pericarpium, Sancho (Fruit bark of Zanthoxylum schinifolium Siebold & Zucc.) extracts

Wasabi Folium (WF) extract: Wasabi Folium, Wasabiyeob (Leaf of Wasabia japonica (Miq.) Matsum.) extracts

Dried Jihwang (RR) extract: Rehmanniae Radix, Jihwang (Root of Rehmannia glutinosa (Gaertn.) Libosch. ex Steud.) extracts

### 1. Preparation of Sole Extract

### (1) Sancho Extract (ZP)

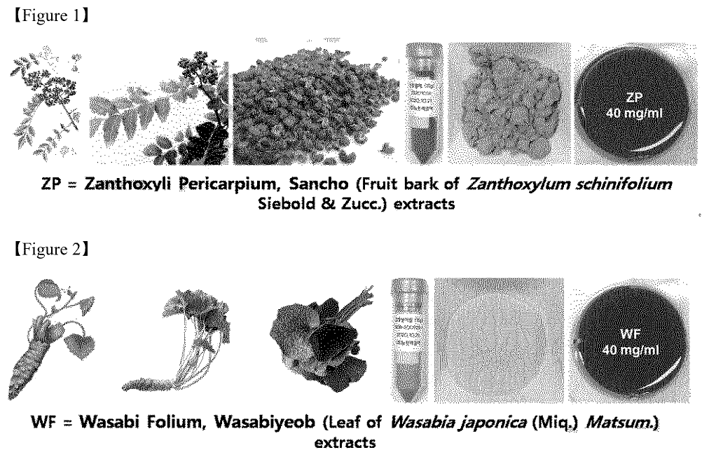

Zanthoxyli Pericarpium (Sancho) was obtained from an herbal medicine store in Suwon, Korea. Extraction was performed at 100° C. for 8 hours using 8 times water (V/V) as a solvent, and then extraction was performed at 100° C. for 4 hours using 4 times water as a solvent. Afterwards, it was filtered and concentrated to 20-25 brix at 60-65° C., and then spray-dried under the conditions of inlet 175° C. and outlet 80° C. to prepare Sancho extract as light reddish brown powders (FIG. 1).

200 mg of each of the Sancho extracts prepared above was weighed, 20 ml of water was added thereto, and the mixture was boiled in a bath at 70° C. for 1 hour. After filtering, it was used as a sample. It was eluted by gradient method at a flow rate of 0.5 ml/min for 65 minutes and measured at an absorption wavelength of 265 nm. Capcell pak C18 (4.6×250 mm, 5 μm) from Phenomenex (USA) was used as a column, and A: water with 0.05% of TFA and B: ACN were used as mobile phases at 30° C. As a result of the experiment, it was confirmed that the content of syringin, an indicator component of Sancho extract, was 0.5 to 10.0 mg/g.

### (2) Wasabi Leaf (WF) Extract

Wasabi leaves were pulverized and extracted at 100° C. for 8 hours using 18 times water (V/V) as a solvent. Afterwards, it was filtered and concentrated to 20-25 brix at 60-65° C., and then spray dried under the conditions of inlet 175° C. and outlet 75° C. to prepare wasabi leaf extract as yellowish brown powders (FIG. 2).

500 mg of each wasabi leaf extract prepared above was weighed, 50 ml of 50% CAN was added thereto, and the mixture was boiled in a bath at 75° C. for 1 hour. After filtering, it was used as a sample. The standard product was prepared and used at a concentration of mg/ml in 50% CAN. It was measured at an absorption wavelength of 228 nm, using a CAPCELL PAK C18, 4.6×250 mm, 5 μm column, and 01% TFA and ACN as developing solvents, at a rate of 0.5 ml/min. As a result, it was confirmed that the content of sinigrin, an indicator component of wasabi leaf extract, was 2.2 to 6 mg/g, and the content of isovitexin was 0.1 to 3.0 mg/g.

### (3) Dried Jihwang (RR) Extract

Dried Rehmanniae Radix was provided by an herbal medicine store in Suwon, Korea. After pulverizing the sample, extraction was performed at 100° C. for 8 hours using 8 times water (V/V) as a solvent, and then extraction was performed at 100° C. for 4 hours using 5 times water (V/V) as a solvent. Afterwards, it was filtered and concentrated to 20-25 brix at 60-65° C., and then spray dried under the conditions of inlet 175° C. and outlet 80° C. to prepare dried Jihwang extract as light brown powders.

### 2. Preparation of the Composite

A composite was prepared by mixing the Sancho extract (ZP) and Wasabi leaf extract (WF) prepared in (1) above in the weight ratio shown in Table 1 below.

TABLE 1

| Weight ratio of Sancho | | Wasabi leaf | |
|---|---|---|---|
| extract (ZP):Wasabi | Sancho extract | extract | Total weight of |
| leaf extract (WF) | (ZP) (g) | (WF) (g) | the composite (g) |
| 1:1 | 50 | 50 | 100 |
| 1:2 | 33 | 67 | 100 |
| 1:4 | 20 | 80 | 100 |
| 1:6 | 14 | 86 | 100 |
| 1:8 | 11 | 89 | 100 |
| 2:1 | 67 | 33 | 100 |
| 4:1 | 80 | 20 | 100 |
| 6:1 | 86 | 14 | 100 |
| 8:1 | 89 | 11 | 100 |

### II. Efficacy Evaluation

### 1. Establishment of Animal Model

In this study, as part of the development of functional food ingredients derived from natural products to improve periodontitis and periodontal bone damage, three types of natural product extracts, dried rehmanniae radix, wasabi leaf, and Sancho extracts, that are well known for various physiological activities, including anti-inflammatory and antioxidant effects were compared and explored using a tooth neck ligation rat periodontal disease (experimental periodontitis; EPD) model to investigate the efficacy against periodontitis and periodontal bone loss.

Specifically, a total of 121 SPF/VAF Outbred Crl:CD [SD] male rats (OrientBio, Seungnam, Korea) were obtained, and after acclimatization for 7 days, experimental animals with a constant body weight were selected, and 10 animals per group were divided and tested in 7 groups below (normal medium control group: average 248.90±8.23 g, 236.00˜261.00 g; EPD-induced group: average 248.14±9.85 g, 219.00˜272.00 g).

Group separation (total of 6 groups; 10 animals per group):
i. Normal control group (control group without tooth neck ligation; oral administration of sterile distilled water)
ii. EPD control group (tooth neck ligation medium control; oral administration of sterile distilled water)
iii. IND-administered EPD group (a group orally administered 5 mg/kg IND (Indomethacin) after tooth neck ligation)
iv. Dried Jihwang-administered EPD group (a group orally administered 200 mg/kg of dried Rehmanniae radix extract after tooth neck ligation)
v. Wasabi leaf-administered EPD group (a group orally administered 200 mg/kg of wasabi leaf extract after tooth neck ligation)
vi. Sancho-administered EPD group (a group orally administered 200 mg/kg of Sancho extract after tooth neck ligation)

To induce periodontal disease, experimental animals were acclimatized to the laboratory environment and then were subjected to general anesthesia by inhaling the mixed gas of 3% isoflurane (Hana Pharm. Co., Hwasung, Korea), 70% N₂O and 28.5% O₂ using a rodent inhalation anesthetic device (Surgivet, Waukesha, WI, USA) and a ventilator (Model 687, Harvard Apparatus, Cambridge, UK). Ligation was performed on the tooth neck of the left incisor using 3-0 nylon suture to induce periodontitis and gingival disease. Meanwhile, in the normal medium control group, only the neck of the incisor was checked and no ligation was performed.

Three natural product extracts above were dissolved in sterile distilled water at a concentration of 40 mg/ml, and administered orally using a metal sonde attached to a 5 ml syringe at a dose of 5 ml/kg (200 mg/kg) once daily for 10 days starting 24 hours after tooth neck ligation. IND was also dissolved in sterilized distilled water at a concentration of 1 mg/ml and administered orally using a metal sonde attached to a 5 ml syringe at a dose of 5 ml/kg (5 mg/kg) once daily for 10 days starting 24 hours after tooth neck ligation. In the normal medium control group and the EPD control group, only sterilized distilled water as a medium was administered orally at the same dose and frequency instead of IND or natural product extract (Table 2).

TABLE 2

| | | |
|---|---|---|
| Group | EPD | Dosage (mg/kg/day) |

Control group

| | | |
|---|---|---|
| Intact | No ligature | Oral administration of distilled water (5 ml/kg) |
| EPD | Incisior ligature | Oral administration of distilled water (5 ml/kg) |

Reference

| | | |
|---|---|---|
| IND | Incisior ligature | 5 mg/kg (oral administration of IND) |

Test herbal extracts (200 mg/kg)

| | | |
|---|---|---|
| RR | Incisior ligature | Oral administration of RR extract |
| WF | Incisior ligature | Oral administration of WF extract |
| ZP | Incisior ligature | Oral administration of ZP extract |

EPD = Experimental periodontitis;
IND = Indomethacin

Daily body weight was measured from 1 day before ligation, periodontal bone loss rate was measured on the final sacrifice day, some gingival tissue was extracted, changes in IL-1β, TNF-α, and MDA content were observed. The number of infiltrating inflammatory cells in the gingival tissue, periodontal bone volume (alveolar bone volume), and collagen content in the gums were measured.

### 2. Changes in Periodontal Bone Loss Rate

In the case of the EPD control group, a significant (p<0.01) increase in the exposed area of the incisor root due to periodontal bone loss, that is, an increase in the periodontal bone loss rate, was recognized compared to the normal medium control group. However, a significant (p<0.01) decrease in periodontal bone loss rate was recognized compared to the EPD control group in the Sancho, Wasabi Leaf, IND, and Dried Rehmanniae radix administration groups in this order.

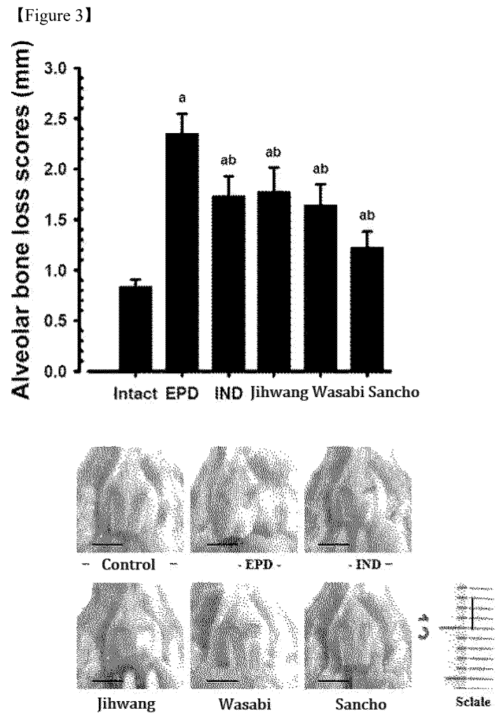

The periodontal bone loss rate in the EPD control group showed a change of 182.55% compared to the normal medium control group, but in the IND, dried Rehmanniae radix, wasabi leaf, and Sancho extract groups, the change was −26.49, −24.53, −30.32, and −48.21%, respectively, compared to the EPD control group (FIG. 3).

### 3. Changes in Periodontal Bone Mass

In the case of the EPD control group, a significant (p<0.01) decrease in periodontal bone mass was recognized compared to the normal medium control group, but a significant (p<0.01) increase in periodontal bone mass was recognized compared to the EPD control group in the order of Sancho, Wasabi Leaf, IND, and Dried Rehmanniae radix administration groups.

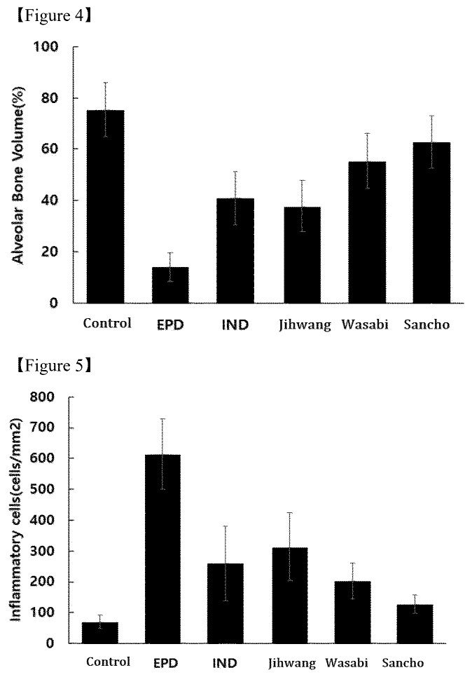

Periodontal bone mass in the EPD control group showed a change of −81.16% compared to the normal medium control group, but in the IND, dried Rehmanniae radix, wasabi leaf, and Sancho extract group, it showed changes of 188.14, 165.61, 290.47, and 341.17%, respectively, compared to the EPD control group (FIG. 4).

### 4. Changes in the Number of Infiltrating Inflammatory Cells in Gingival Tissue

In the case of the EPD control group, a significant (p<0.01) increase in the number of infiltrating inflammatory cells in the gingival tissue was recognized compared to the normal medium control group, but there was a significant (p<0.01) decrease in the number of infiltrating inflammatory cells in the gingival tissue compared to the EPD control group in the order of Sancho, Wasabi Leaf, IND, and Dried Rehmanniae radix administration groups.

The number of infiltrating inflammatory cells in the EPD control group showed a change of 756.82% compared to the normal medium control group, but in the IND, dried Rehmanniae radix, wasabi leaf, and Sancho extract groups, the changes were −57.61, −49.12, −66.91, and −79.10, respectively, compared to the EPD control group (FIG. 5).

### 5. Changes in the Content of Collagen Fibers in Gingival Tissue

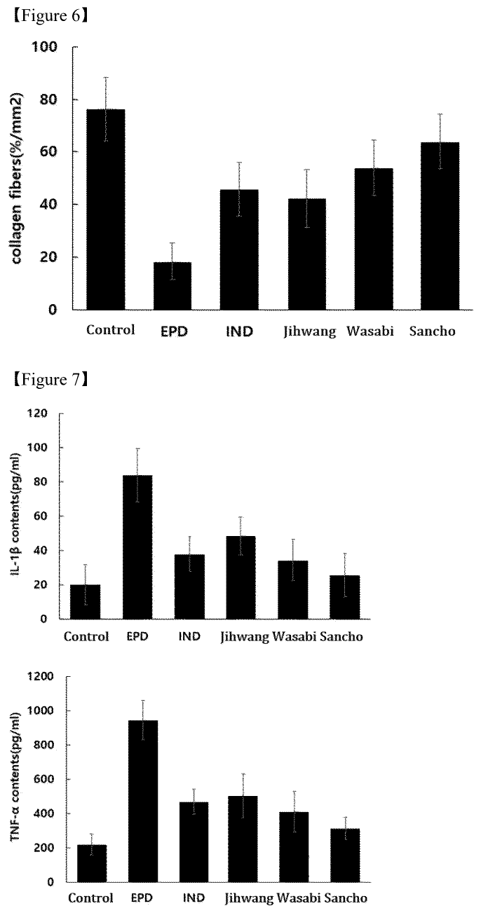

In the case of the EPD control group, a significant (p<0.01) decrease in the proportion of collagen fibers in gingival tissue was recognized compared to the normal medium control group, but a significant (p<0.01) increase in the proportion of collagen fibers in gingival tissue was recognized compared to the EPD control group in the order of Sancho, Wasabi Leaf, IND, and Dried Rehmanniae radix administration groups (FIG. 6).

The number of infiltrating inflammatory cells in the EPD control group showed a change of −75.75% compared to the normal medium control group, but in the IND, dried Rehmanniae radix, wasabi leaf, and Sancho extract groups, there was a change of 148.65, 129.20, 191.72, and 245.59%, respectively, compared to the EPD control group.

### 6. Activity of Inhibiting Inflammatory Cytokine Expression in Gums

### (1) Changes in IL-β1 Content in Gingival Tissue

In the case of the EPD control group, a significant (p<0.01) increase in IL-1β content in gingival tissue was recognized compared to the normal medium control group, but a significant (p<0.01) decrease in IL-1β content was observed compared to the EPD control group in the order of Sancho, Wasabi Leaf, IND, and Dried Rehmanniae radix administration groups, respectively.

IL-1β content in gingival tissue showed a change of 314.86% in the EPD control group compared to the normal medium control group, but in the IND, dried Rehmanniae radix, wasabi leaf, and Sancho extract groups, the changes were −54.75, −42.28, −58.99, and −69.40%, respectively, compared to the EPD control group (top of FIG. 7).

### (2) Changes in TNF-α Content in Gingival Tissue

In the case of the EPD control group, a significant (p<0.01) increase in TNF-α content in gingival tissue was recognized compared to the normal medium control group, but there was a significant (p<0.01) decrease in TNF-α content compared to the EPD control group in the order of Sancho, Wasabi leaf, IND, and Dried Rehmanniae radix administered groups. was recognized, respectively.

The TNF-α content in gingival tissue showed a change of 329.08% in the EPD control group compared to the normal media control group, but in the IND, dried Rehmanniae radix, wasabi leaf, and Sancho extract groups, the changes were −50.46, −46.54, −56.37, and −66.84%, respectively, compared to the EPD control group (bottom of FIG. 7).

### 7. Changes in MDA Content in Gingival Tissue

In the case of the EPD control group, a significant (p<0.01) increase in MDA content in gingival tissue was recognized compared to the normal medium control group, but a significant (p<0.01) decrease in MDA content was observed compared to the EPD control group in the order of Sancho, Wasabi Leaf, IND, and Dried Rehmanniae radix administration groups.

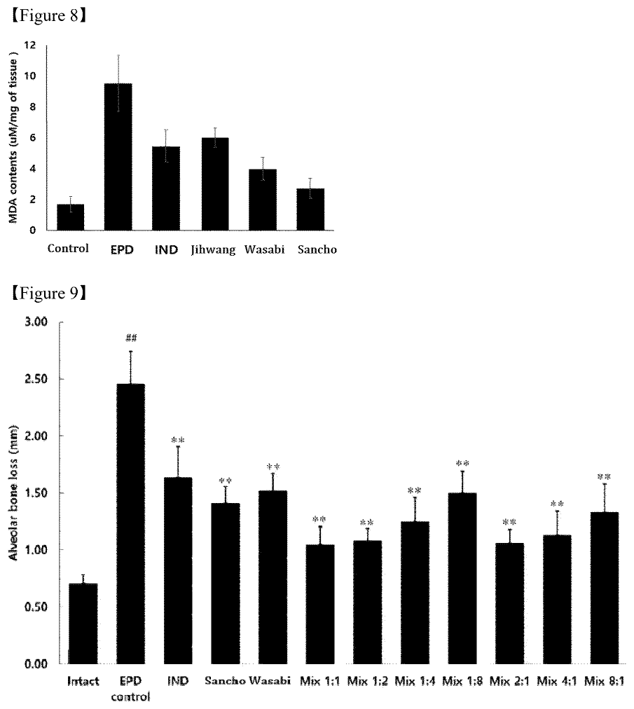

The MDA content in gingival tissue showed a change of 460.41% in the EPD control group compared to the normal media control group, but in the IND, dried Rehmanniae radix, wasabi leaf, and Sancho extract groups, the changes were −42.44, −36.82, −58.10, and −71.23%, respectively, compared to the EPD control group (FIG. 8).

As mentioned above, as part of the development of natural product-derived functional foods and natural pharmaceutical materials to improve periodontitis and periodontal bone damage, the medicinal effects of three types of natural product extracts (dried Rehmanniae radix, wasabi leaf, and Sancho) on periodontitis and periodontal bone loss were investigated through tooth neck ligation using the EPD rat model. As a result of comparative evaluation, the anti-inflammatory effect on gingivitis induced by tooth neck ligation was recognized in the group administered Sancho, wasabi leaf, IND, and dried Rehmanniae radix consistently in the same order, and the effect on inhibition of periodontal bone loss was observed in the same order. Therefore, under the conditions of this experiment, Sancho and wasabi leaf extracts, which showed a relatively better inhibitory effect at a dose of 200 mg/kg than IND 5 mg/kg on gingivitis and periodontal bone loss induced by tooth neck ligation, have been confirmed to have efficacy as an active ingredient derived from natural products for improving periodontitis and periodontal bone damage.

### 8. Confirmation of Efficacy of the Composite

The efficacy of the composite of Sancho extract (ZP) and wasabi leaf extract (WF), which were confirmed to have excellent effects, on periodontitis and periodontal bone loss was compared and examined using a tooth neck ligation rat periodontal disease (experimental periodontitis; EPD) model.

To induce periodontal disease, experimental animals were acclimatized to the laboratory environment and then were subjected to general anesthesia by inhaling the mixed gas of 3% isoflurane (Hana Pharm. Co., Hwasung, Korea), 70% N₂O and 28.5% O₂ using a rodent inhalation anesthetic device (Surgivet, Waukesha, WI, USA) and a ventilator (Model 687, Harvard Apparatus, Cambridge, UK). Ligation was performed on the tooth neck of the left incisor using 3-0 nylon suture to induce periodontitis and gingival disease. Meanwhile, in the normal medium control group, only the neck part of the incisor was checked and no ligation was performed.

ZP (Sancho extract) and WF (wasabi leaf extract) alone and 9 types of ZP:WF composites as shown in Table 1 (ZP:WF=1:1, 1:2, 1:4, 1:6, 1:8, 2:1, 4:1, 6:1 and 8:1 (g/g)) were used as the experimental substances. The administered dose of the test substance was set to the same total amount of 100 mg/kg based on the drug efficacy exploration study of the present inventors. The test substance was dissolved in sterile distilled water and administered orally.

The above nine types of composites were administered orally at a dose of 100 mg/kg each, once daily for 10 days starting 24 hours after tooth neck ligation, using a metal sonde attached to a 5 ml syringe. The IND was also administered at a dose of 1 mg/ml. IND was also dissolved in sterilized distilled water at a concentration of 1 mg/ml and administered orally using a metal sonde attached to a 5 ml syringe at a dose of 5 ml/kg (5 mg/kg) once daily for 10 days starting 24 hours after tooth neck ligation. In the normal medium control group and the EPD control group, only sterilized distilled water as a medium was administered orally at the same dose and frequency instead of IND or the above composites.

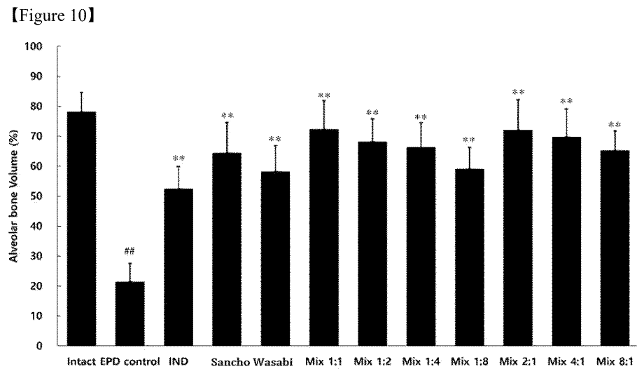

The results of measuring the change in periodontal bone loss rate by measuring the periodontal bone loss rate on the final sacrifice day are shown in FIG. 9, and the results of measuring the change in periodontal bone volume (Alveolar bone volume) are shown in FIG. 10.

As a result of the experiment, the group administered 100 mg/kg of ZP:WF 1:4, 1:2, 1:1, 2:1, 4:1, and 8:1 (g/g) composite composition showed a lower periodontal bone loss rate compared to the group administered the same dose of ZP and WF alone, meaning a synergistic effect due to the combination of the two components. In particular, the group administered 100 mg/kg of ZP:WF 1:2, 1:1, 2:1 and 4:1 (g/g) composite composition showed a remarkable synergistic effect (FIG. 9).

In addition, as a result of measuring changes in periodontal bone volume (Alveolar bone volume), the group administered 100 mg/kg of 1:4, 1:2, 1:1, 2:1, 4:1, and 8:1 (g/g) composite composition showed a higher periodontal bone mass than the group administered the same dose of ZP and WF alone, showing a synergistic effect of the combination of the two components. In particular, the group administered 100 mg/kg of the ZP:WF 1:2, 1:1, 2:1 and 4:1 (g/g) composite composition showed a remarkable synergistic effect (FIG. 10).

1. A composition for preventing, improving or treating periodontal disease comprising a combination of Sancho extract (ZP) and wasabi leaf extract (WF) as an active ingredient.

2. The composition according to claim 1, wherein the weight ratio of the Sancho extract to the wasabi leaf extract is 1:4 to 8:1 (ZP:WF).

3. The composition according to claim 1, wherein the weight ratio of the Sancho extract to the wasabi leaf extract is 1:2 to 4:1 (ZP:WF).

4. The composition according to claim 1, wherein the weight ratio of the Sancho extract to the wasabi leaf extract is 1:2 to 2:1 (ZP:WF).

5. The composition according to claim 1, wherein the wasabi leaf extract contains 1.0 to 27.7 mg/g of sinigrin or 0.1 to 3.0 mg/g of isovitexin.

6. The composition according to claim 1, wherein the extract is extracted using any one selected from the group consisting of water, lower alcohols having 1 to 4 carbon atoms, and mixtures thereof as an extraction solvent.

7. The composition according to claim 1, wherein the periodontal disease is gingivitis or periodontitis.

8. The composition according to claim 1, wherein the Sancho extract contains 0.5 to 10.0 mg/g of syringin.

9. The composition according to claim 1, wherein the composition is a food composition for preventing or improving periodontal disease.

10. The composition according to claim 1, wherein the composition is a pharmaceutical composition.

11. A health functional food containing the composition of claim 9.

12. The composition according to claim 1, wherein the composition is a toothpaste composition.

13. A method for improving or treating periodontal disease in a subject in need thereof comprising administering to said subject an effective amount of a combination of Sancho extract (ZP) and wasabi leaf extract (WF).

14. The method according to claim 13, wherein the weight ratio of the Sancho extract to the wasabi leaf extract is 1:4 to 8:1 (ZP:WF).

15. The method according to claim 13, wherein the weight ratio of the Sancho extract to the wasabi leaf extract is 1:2 to 4:1 (ZP:WF).

16. The method according to claim 13, wherein the periodontal disease is gingivitis or periodontitis.
